(19) United States
(12) Patent Application Publication
(10) Pub. No.: US 2026/0135003 A1
(43) Pub. Date: May 14, 2026

(54) HUMAN OMIC-DRIVEN MACHINE LEARNING METHODS AND SYSTEMS FOR EVALUATING PATIENT SAFETY

(71) Applicant: Janssen Research & Development, LLC, Raritan, NJ (US)

(72) Inventors: Evan Harris Baugh, River Edge, NJ (US); Brian Scott Mautz, Norristown, PA (US); Kaitlin Ann Hood, Philadelphia, PA (US); Alvaro Emilio Ulloa Cerna, Danville, PA (US)

(73) Assignee: Janssen Research & Development, LLC, Raritan, NJ (US)

(21) Appl. No.: 19/388,741

(22) Filed: Nov. 13, 2025

Related U.S. Application Data
(60) Provisional application No. 63/720,008, filed on Nov. 13, 2024.

(51) Int. Cl. G16H 50/30; G06N 3/09

(52) U.S. Cl. CPC G16H 50/30; G06N 3/09

(57) ABSTRACT

A computer-implemented method of estimating a risk associated with patient safety is disclosed. One or more computers execute processing comprising receiving a training data set comprising drug-associated patient safety data and human omics data. One or more probabilistic features from the training data set are identified, where the one or more probabilistic features comprise human omics features associated with a drug therapeutic. A ground truth label associated with the drug therapeutic and comprising a positive or a negative patient safety outcome corresponding to the one or more probabilistic features is derived. The method trains one or more machine learning models in accordance with the ground truth label and applies the trained machine learning models to a patient dataset of human omics data to generate a calculated indication of a positive or a negative patient safety outcome. The method takes one or more actions in response to the calculated indication.

### CROSS REFERENCE TO RELATED APPLICATIONS

This application claims the benefit of U.S. Provisional Application No. 63/720,008, filed on Nov. 13, 2024. The contents of that application are incorporated by reference herein.

### BACKGROUND

The evaluation of gene target safety is a crucial step in the development of new therapeutic treatments including gene therapies and precision medicines.

### SUMMARY

This disclosure relates generally to machine learning technologies for making automated safety risk assessments for targeted gene therapies and precision medicines. Thorough safety evaluation of new drugs is significantly constrained by time and monetary costs required to generate, curate, and interpret diverse data types such as genomics, proteomics, and clinical information, and to integrate these findings with the scientific and medical research literature. Multidisciplinary teams are typically required to perform these data analytics tasks, further straining resources, budgets, and time. As research and development teams attempt to understand underlying biology of the new drug molecules that are generated or found, delays in development processes can occur. Additionally, for novel gene targets, the lack of sufficient data to evaluate safety can adversely impact proper safety evaluations.

As safety concerns account for approximately 30% of clinical trial failures, a computational solution to guide and standardize safety evaluation in the preclinical space is needed. Previously, algorithms evaluating adverse events (AEs) have centered on drug and drug-feature derived risks. However, such information is not necessarily useful in initial stages when molecules are still under development. Human genetics and human omics data has been shown to help increase the relative success of new drugs. For example, human genetic evidence alone already increases clinical success rates by 2-8×; while further integrated multi-omics data can improve risk estimation, enabling rapid, scalable, and standardized safety evaluation.

Advances in analytical methods such as artificial intelligence (AI) and machine learning (ML) combined with the increasing availability of diverse omics data provides the opportunity to perform safety evaluations in a timely manner. ML models can be built on existing datasets either as known outcomes or feature spaces, or both, to help estimate risk. ML models can learn which Omics features contribute to estimated known AEs and can then guide overall safety evaluations, including ranking risk concerns. Trained on known examples, these models can be further applied to model safety outcomes for novel gene targets, when Omics and other gene target features are available, even if published research literature on such novel targets is sparse or unavailable. Given the novelty of such omics-driven ML methods and systems to evaluate patient safety of new drugs, it can also be beneficial to evaluate the ability of the ML models to estimate the correct risks and compare the outcomes of the ML models relative to other known evaluation methods.

Embodiments of the present disclosure leverage human omics data to develop a gene-based machine learning (ML) model that estimates organ category (OC) level risk to systematize safety assessments and prioritize drug targets in the pre-clinical space.

A computer-implemented method of estimating a risk associated with patient safety is disclosed. One or more computers execute processing comprising receiving a training data set comprising drug-associated patient safety data and human omics data. One or more probabilistic features from the training data set are identified, where the one or more probabilistic features comprise human omics features associated with a drug therapeutic. A ground truth label associated with the drug therapeutic and comprising a positive or a negative patient safety outcome corresponding to the one or more probabilistic features is derived. The method trains one or more machine learning models in accordance with the ground truth label and applies the trained machine learning models to a patient dataset of human omics data to generate a calculated indication of a positive or a negative patient safety outcome. The method takes one or more actions in response to the calculated indication.

Some embodiments of the present disclosure describe a computer-executable deep learning network to estimate a patient safety risk, where the computer-executable deep learning network is stored in a non-transitory computer readable medium and configured to execute on one or more computer processors. A training data set comprising drug-associated patient safety data and human Omics data is communicatively connected to a feature space having one or more probabilistic features. The probabilistic features comprise one or more human omics features associated with a drug therapeutic. A pre-trained machine learning analyzer comprising one or more machine learning models is communicatively coupled to the feature space and the training database. The pre-trained machine learning analyzer is configured to receive one or more gene targets and generate one or more calculated indications. The machine learning analyzer is trained by first deriving a ground truth label comprising a positive or a negative patient safety outcome corresponding to the one or more probabilistic features of the human omics data, wherein the ground truth label is associated with the drug-associated patient safety data of the drug therapeutic. The one or more ML models are then trained using the training data set in accordance with the ground truth label.

Some embodiments of the present disclosure describe a non-transitory computer readable medium configured for storing one or more computer readable instructions that, upon execution by one or more processors, perform operations including training at least one machine learning model using one or more databases comprising one or more target genes, one or more drug therapeutics associated with the one or more target genes, adverse event data associated with the one or more drug therapeutics and the one or more target genes, and a plurality of human omics features associated with the one or more target genes derived from human omics data. Next, one or more calculated indications of a positive or a negative patient safety outcome for one or more gene targets are generated by applying the trained one or more machine learning models to a patient dataset of human omics data.

These and other aspects of the present disclosure are more fully detailed below. This summary is provided to introduce a selection of concepts in a simplified form that are further described below in the Detailed Description. This summary is not intended to identify key features or essential features of the claimed subject matter, nor is it intended to be used to limit the scope of the claimed subject matter. Various objects, features, aspects, and advantages of the inventive subject matter will become more apparent from the following Detailed Description, along with the accompanying drawing figures. In the figures, like numerals represent like components.

### BRIEF DESCRIPTION OF THE DRAWINGS

For the purposes of illustration only, several aspects of embodiments of the invention are described in reference to the following figures. In the following figures, the same number represents the same type of element in all drawings.

FIG. 1 illustrates example diagrams depicting generation of training data and training features in accordance with an embodiment of the disclosure.

FIG. 1A illustrates a flow diagram depicting an overview of a gene-based machine learning algorithm in accordance with embodiments of the present disclosure.

FIG. 2 is a flow diagram illustrating a method for training a gene-based patient safety risk assessment deep learning network. The illustrated method is in accordance with an embodiment of the disclosure.

FIG. 3 illustrates a flow diagram depicting the operation of the machine learning model in accordance with some embodiments of the present disclosure.

FIG. 4 illustrates flow diagrams depicting the training and testing operations of a gene-based patient safety risk assessment deep learning network in accordance with some embodiments of the present disclosure.

FIG. 5 illustrates an example flow diagram for a computer-implemented method for estimating a risk associated with patient safety in accordance with an embodiment of the present disclosure.

FIG. 6 shows an example flow diagram of filtering operations used to generate training labels for development of a machine learning model for a target risk assessment profile in accordance with an embodiment of the present disclosure.

FIG. 7A shows an example of a user interface of an application for finding risk scores for one or more gene targets in accordance with an embodiment of the present disclosure.

FIG. 7B shows an example of a user interface for customizing risk scores for one or more gene targets in accordance with an embodiment of the present disclosure.

FIG. 8 shows an example of a computer system, one or more of which may be used to implement one or more of the apparatuses, systems, and methods illustrated herein.

While the embodiments are described with reference to the above drawings, the drawings are intended to be illustrative, and other embodiments are consistent with the spirit, and within the scope, of the disclosure.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the disclosure to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

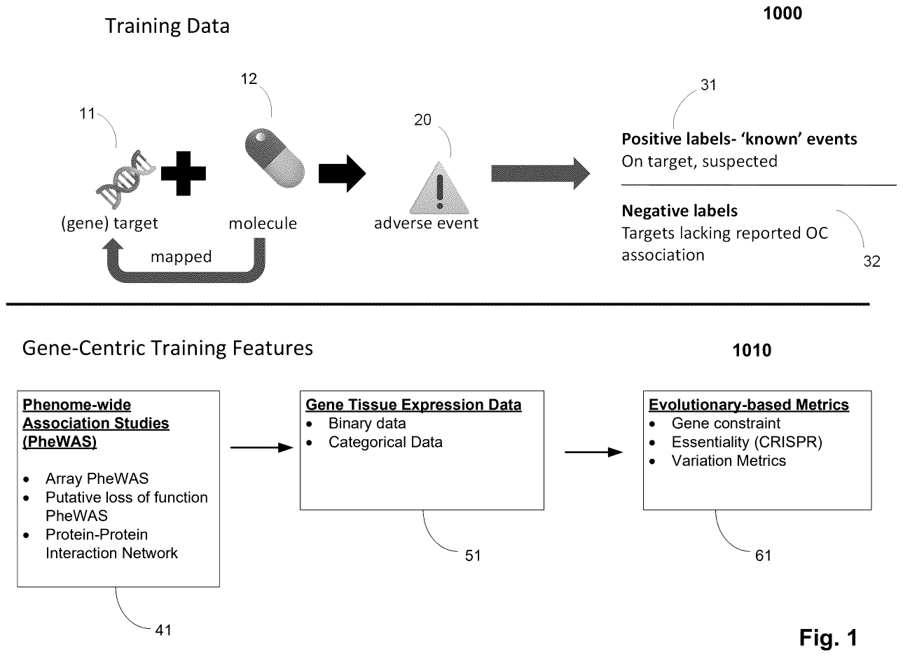

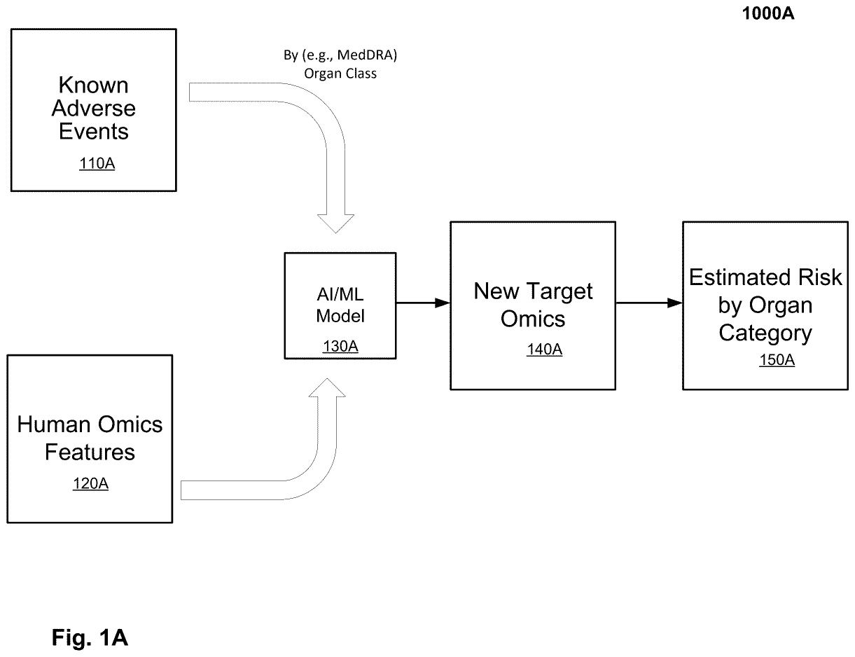

FIG. 1 illustrates example diagrams for depicting generation of training data (1000) and training features (1010) in accordance with an embodiment of the disclosure. In one embodiment of the disclosure, a database of gene-centric known adverse events (AE) and human omics features is developed. In one example, one or more clinical intelligence databases, such as the OFF-X translational safety intelligence platform, compiles drug-associated AE data from pre-clinical through post-marketing drug development and can be used as a resource. In some examples disclosed herein, drugs 12 in the OFF-X platform (or similar database) can be selected that are small molecule inhibitors and mapped to their primary gene targets 11. In some embodiments, confirmed and suspected AE tags 20 for each drug-gene combination can be kept as positive (e.g., “known”) labels, while drug-gene combinations without a label in that AE category are kept as negative labels (e.g., lacking reported or suspected AEs). One having ordinary skill in the art will understand the data sources and standards described herein to be exemplary, and any source of data and/or regulatory standard or terminology may be alternately or additionally used as appropriate.

Human omic features, in some embodiments, can include genetic Phenome-wide Association Studies (PheWAS) 41, gene tissue expression data 51, and evolutionary-based metrics 61, or any subset or combination thereof. In some embodiments, gene constraint and tolerance to loss of function metrics can be estimated from variant annotations from UK Biobank (UKB) and associated electronic health records (EHR) data, and other public or non-public resources (e.g., gnomAD genome aggregation database, LoFTool, GeVIR, RVIS). Variant and gene-level phenome-wide association statistics can also be derived from array and whole genome sequence data (e.g., UKB). In some embodiments, causal inference statistics are derived from co-occurrence of array gene-level phenome-wide association statistics (e.g., from UKB) and expression quantitative trait loci, (e.g., GTEx expression quantitative trait loci).

In some embodiments, essentiality scores are based on CRISPR/Cas-9 data from PICKLE v3 database, while cell- and tissue-specific gene expression data are derived from Human Body Map and FANTOM5. Gene-level descriptive metrics for gene product protein localization and metabolism are obtained from MSigDB and DGIdb, with gene-level disease association inheritance modality from MedGen. In some embodiments, protein-protein interaction network information and properties are obtained from Protein InteraCtion KnowLedgebasE (PICKLE). Standardized centrality metrics (eigenvector, closeness, degree, betweenness) can be generated directly from PICKLE.

FIG. 1A illustrates a flow diagram 1000A depicting an overview of a gene-based machine learning algorithm to aid in target safety evaluation, in accordance with embodiments of the present disclosure. In one embodiment of technologies disclosed herein, target risk assessment profiling is performed by developing a database of gene-centric known adverse events (AE) 110A and human omics features 120A. In one example implementation of 110A, a database compiling drug-associated AE data from preclinical through post-marketing can be used as a source of positive and negative AE labels. In this example implementation, drugs in the database that are small molecule inhibitors are selected and mapped to their primary gene targets. Confirmed and suspected AE tags for each drug-gene combination can be maintained as positive (“known”) labels, while drug-genes without a label in that AE category can be kept as negative labels. In one example, to avoid overly sparse data, specific AEs (e.g., heart attack, depression) are collapsed into their corresponding organ category (OC) terms derived from the MedDRA terminology (e.g., cardiac, psychiatric). In some examples, 19 organ categories (OCs) derived from the MedDRA or Medical Dictionary for Regulatory Activities from the ICH (International Council for Harmonization of Technical Requirements for Pharmaceuticals for Human Use) are used. MedDRA is a clinically validated international medical terminology dictionary-thesaurus used by regulatory authorities and the biopharmaceutical industry during the regulatory process, from pre-marketing (clinical research phase 0 to phase 3) to post-marketing activities (pharmacovigilance or clinical research phase 4), and for safety information data entry, retrieval, evaluation, and presentation. MedDRA can also be used as an adverse event classification dictionary.

Human omics features 120A include gene constraint metrics, genetic phenome-wide associations, loss of function profiling, gene expression (e.g., including data derived from e.g., Human Body Map, FANTOM5), and protein interaction network properties.

In the example AI/ML model 130A shown in FIG. 1A, the system is presumed to be fully trained using the database's AE data 110A and human omics features 120A and fine-tuned for end-use in generating estimated risks or otherwise making inferences relevant to a particular task (e.g., risk score(s) for a particular target gene and drug). New target omics can be developed in 140A, where every OC estimate can be dissected into feature contributions to suggest which properties of the gene target confer or correlate with risk estimates. The target risk assessment profile machine learning model(s) 130A, in conjunction with new target omics 140A, generates 19 Organ Category risk scores in 150A, along with a weighted overall gene-based score, to guide risk evaluation and prioritization for over 15,000 protein-coding human genes. One having ordinary skill in the art will appreciate that in other implementations of the systems and methods described herein, an alternate number or type of risk score categorization may be used.

In one example, a multipronged approach can be used to validate the risk scores in 150A. First, risk scores 150A for gene-targets of drugs withdrawn from the market can be compared to those still available, including specification of withdrawals due to death or toxicity alone. Second, top OC risk scores for the gene targets can be aligned with genes (e.g., 70-100 genes) from a standard safety panel to validate the extent to which AI/ML model 130A estimates these known risks for these safety targets. Next, a comparison of risk scores 150A output from AI/ML model 130A can be run against expert-curated literature safety information (via confidence scores) to highlight consistency and difference between the disclosed gene-based machine learning algorithm and more standard drug safety practices.

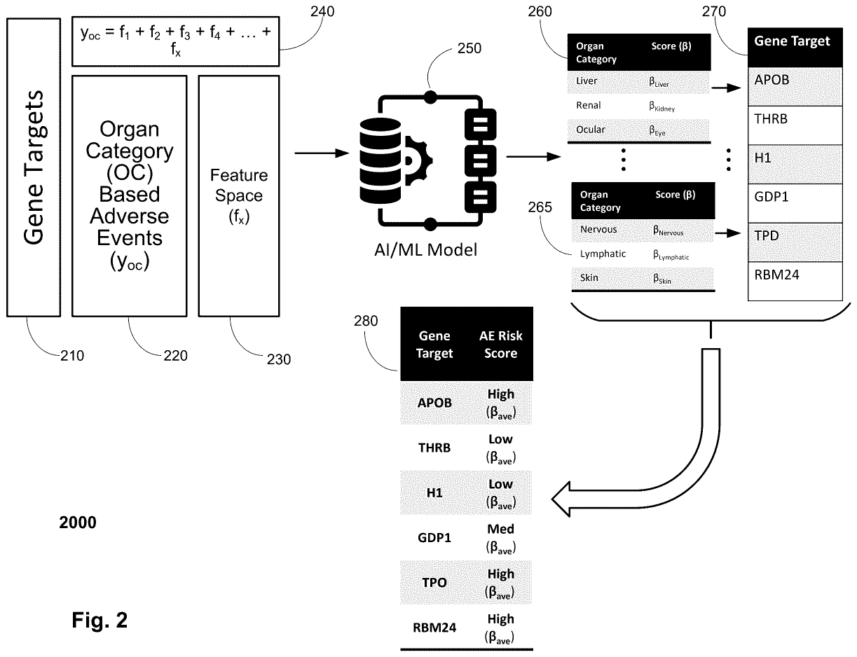

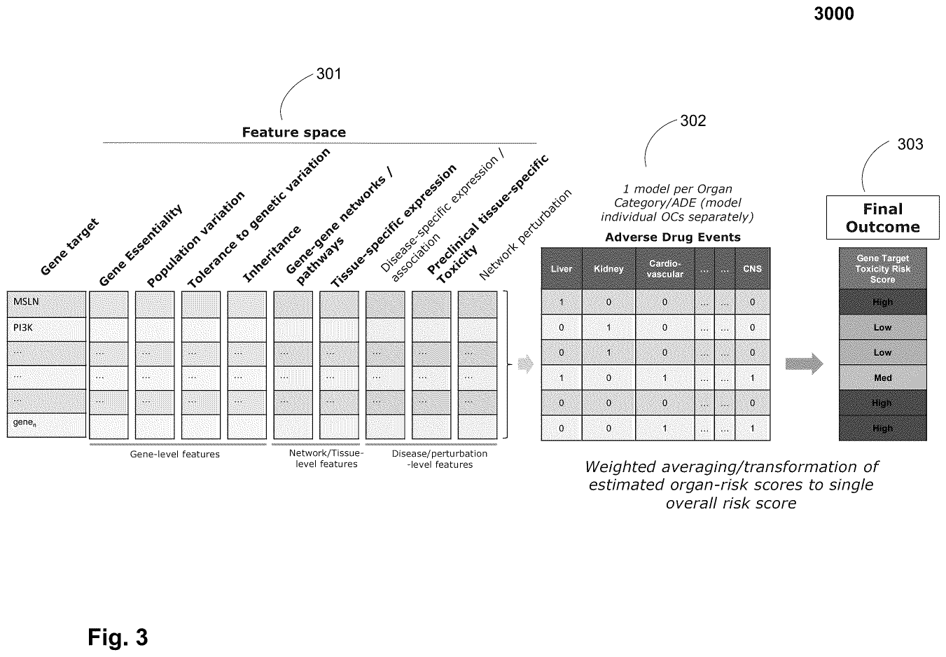

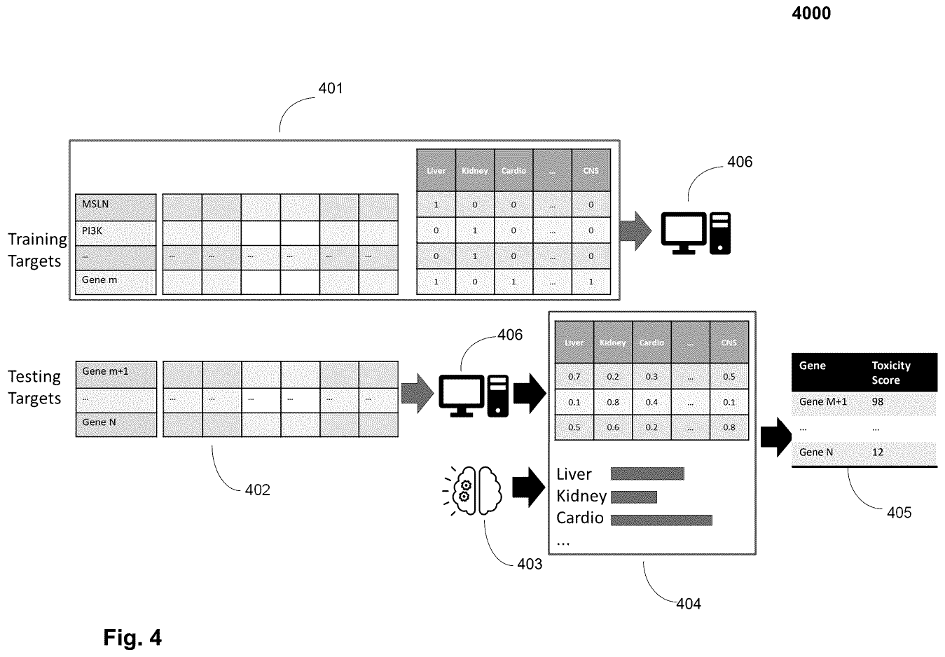

FIG. 2 is a flow diagram 2000 illustrating a method for training a gene-based patient safety risk assessment deep learning network. The illustrated method is in accordance with an embodiment of the disclosure. FIG. 3 illustrates a flow diagram 3000 depicting the operation of a gene-based patient safety risk assessment deep learning network (e.g., machine learning model) in accordance with some embodiments of the present disclosure. FIG. 4 illustrates two flow diagrams 4000 depicting the training and testing operations of a gene-based patient safety risk assessment deep learning network in accordance with some embodiments of the present disclosure. The following discussion references FIGS. 2, 3, and 4 together below.

First, a list of drugs (e.g., mostly small-molecule inhibitors), and their primary gene targets 210 can be provided, as described herein with respect to some embodiments. In addition, a list of organ category-based adverse events 220 can also be obtained. While, in an example embodiment, these items are included in OFF-X, other implementations may obtain data from other translational safety databases. AEs can be aggregated into MedDRA-based organ category (OC) labels (e.g., cardiac), as specific AEs (e.g., heart attack) can be too sparse to yield robust estimates.

This is also shown at 401 in FIG. 4, where individual gene targets used for training as listed, as well as specific known AEs for each known gene target (along with associated features, where a feature space is shown at 401 but features are not explicitly listed). Feature space 230, in some embodiments, can include a database of approximately 329 binary- and quantile-scaled gene-centric features of human omics data, although smaller (e.g., 142 features) or larger feature spaces can alternatively be used. As an example, the features in feature space 230 can include: gene constraint metrics, variant counts, essentiality scores, tolerance to loss of function metrics, variant and gene-level phenome-wide association statistics (from, e.g., biobanks and disease-cohorts), gene expression, and protein interaction networks. In addition, standardized centrality metrics (eigenvector, closeness, degree, betweenness) can be generated directly from protein interaction network information.

The human omics features relevant to the gene targets can in some embodiments comprise one or more gene-level features (loss of function associations, constraint metrics, gene product localization and metabolism, disease association inheritance modality), network-level features (organ category specific associations of proteins in target network, centrality measures), tissue-level features (sub-tissue, average tissue, and average organ levels expression), cell type features (cell type average expression and cross-sample expression quantification), disease-level features (diagnostic-code derived diseases/risks hierarchically categorized into organ classes for quantitative traits, binary traits, and co-occurrence of binary traits with expression derived quantitative traits), or perturbation-level features (e.g., CRISPR scores, variant counts).

The features of feature space 230 can be integrated with the AE data to form OC-associated adverse events and their associated relevant features in equation 240. Thus, in the trained model, a plurality of gene targets (which can be gene targets used in the training set but need not be) may be associated with one or more features in feature space 301. Gene targets 301 are also associated with adverse drug events 302. As discussed above and shown at 302, one ML is trained for each organ category (OC), and individual OCs are modeled separately, for a total of 19 organ system classifiers (depicted as a computer icon at 406 in FIG. 4).

In one example implementation, the software and specific regression method/function in that package used is scikit-learn v1.0.1, although other regression methods (such as, e.g., XGBoost (extreme gradient boosting)) can be used. In the example implementation, parameters are passed to the logistic regression methods and/or additional functions added to the training model are as follows:
Scikit-learn v1.0.1 (see, e.g., https://scikit-learn.org/stable/modules/generated/sklearn.linear_model.LogisticRegression.html)
LogisticRegression (C=1, class_weight=‘balanced’)
Additional parameters: penalty=′12′, dual=False, tol=0.0001, C=1.0, fit_intercept=True, intercept_scaling=1, class_weight=None, random_state=None, solver=‘lbfgs’, max_iter=100, multi_class=‘auto’, verbose=0, warm_start=False, n_jobs=None, l1_ratio=None

In one example implementation, 19 OC-specific logistic regression models are trained using 5-fold cross validation, where missing data can be mean imputed using scaled values. Performance was reported as the average across the 5 folds with a 95% confidence interval. For logistic regression models, the average AUC ranged from 0.594 for Hepatobiliary disorders to 0.663 for Cardiac disorders.

In some example implementations, LR can provide similar performance to alternative regression methods such as XGB. In addition, differences in performance for ML models across different organ categories can also be attributed to a given set of features available being more informative for certain OCs compared to others.

In this example implementation, AI/ML model 250 thus generates 19 OC risk scores (examples shown in 260 and 265 for 3 exemplary OCs per gene target) for each gene target 270 based on regression likelihood values, with feature weights providing links to omics inputs contributing to increase or decrease risk. Additionally, a gene-based score 280 can also be generated by taking the average across 19 OC scores, which is also shown as a “Gene Target Toxicity Risk Score” at 303 of FIG. 3. In one example implementation, gene-based scores can be generated for over 15,000 human genes by applying the ML model 250 to genes with feature values outside of the given training set. FIG. 4 shows gene targets being tested at 402, with OC risk scores 404 being generated after the gene targets are evaluated using machine learning model 406 to generate gene target toxicity scores 405 (which can also be ranked into high, medium or low toxicity risk depending on the score value). In some example implementations, at least one drug target (gene target) can be de-risked in an early pre-clinical stage based on the calculated indication. A target is ‘de-risked’ by identifying potential underlying biological explanations for this risk association such that risk can be avoided through drug design, drug delivery, treatment modality, systemic versus localized treatment, tissue- or cell-specific absorption, or other means. Such de-risking can be performed by identifying features that may have a high impact on the risk score and developing risk mitigation strategies for that particular gene target. Expert-curated literature assessments 403 can be used in some example implementations for de-risking and validation of gene target toxicity scores.

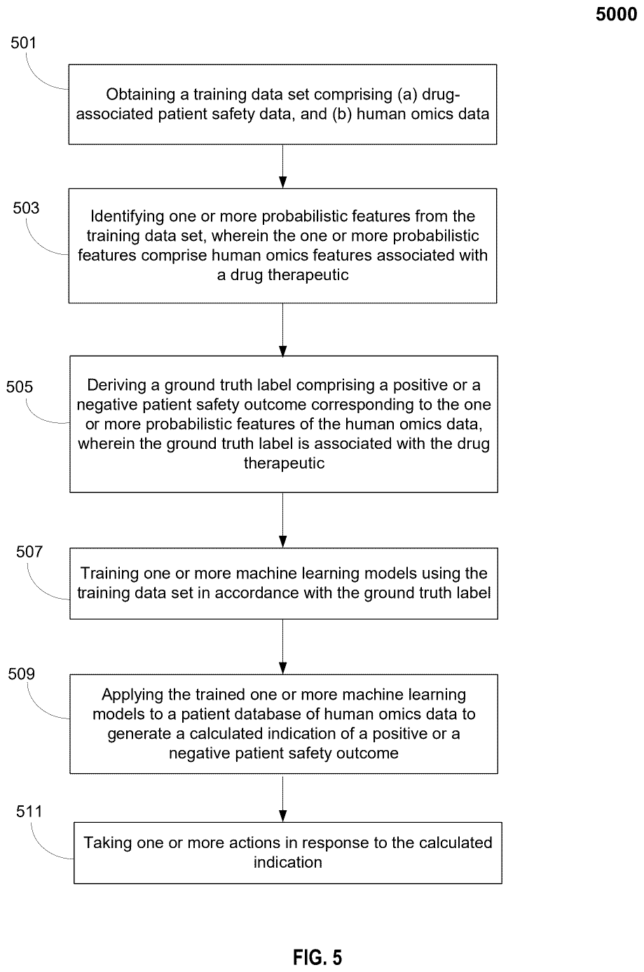

FIG. 5 illustrates an example flow diagram for a computer-implemented method 5000 for estimating a risk associated with patient safety in accordance with an embodiment of the present disclosure, e.g., by training and evaluating machine learning models used to estimate target toxicity. In step 501, a training data set is obtained. The training data set comprises both drug-associated patient safety data as discussed above and human omics data as discussed above.

At step 503, one or more probabilistic features from the training data set are identified, wherein the one or more probabilistic features comprise human omics features associated with a drug therapeutic. The drug-associated patient safety data may contain adverse event information that is converted to binary labels for each gene target indicating toxicity reports for 19 organ categories (OC). Lists of features and Organ Categories derived from MedDRA terminology usable in the computer-implemented method 5000 are provided below in the Example Features and Organ Categories section, though these are merely exemplary and non-limiting.

At step 505, a ground truth label is derived. The ground truth label comprises a positive or a negative patient safety outcome corresponding to the one or more probabilistic features of the human omics data, wherein the ground truth label is associated with the drug therapeutic. In one example implementation, the number of gene targets in the drug-associated patient safety data is reduced to those with at least one positive label (toxicity report) in any organ category and is present in the feature dataset. This yields a total of 18,175 targets for which the number of positive labels range from 349 to 1088 among OCs. Any missing label is imputed as 0, meaning lack of toxicity reported. In this example, negative training samples are restricted to genes containing at least one positive label in any OC while lacking a positive label for the specific OC classifier being trained to ensure negative labels are applied only to genes with sufficient opportunity to have had AEs observed.

At step 507 one or more machine learning models are trained using the training data set in accordance with the ground truth label. In the example implementation described above, the targets can be split into 5 randomly selected folds. For each fold and OC, machine learning (ML) models can be fitted for any combination of feature sets. In one example, the ML model pipeline comprises: a Quantile transformer (scales values assigning it to its quantile value), followed by mean imputation, and a Logistic Regression (LR) classifier. The LR classifier is set to have balanced class weights, meaning that it penalizes the model for misclassification of the minority class.

At step 509, the trained one or more machine learning models are applied to a patient database of human omics data to generate a calculated indication of a positive or a negative safety outcome. For each fold, in the example implementation described above, a set of scores for each organ category can be obtained, and the average performance metric across the five folds can be reported with a 95% confidence interval. In addition, various metrics are collected, including the area under the receiver operating characteristics curve (AUROC), the area under the precision-recall curve (AUPRC), and threshold dependent metrics such as accuracy, sensitivity, specificity, positive predictive value (PPV), and negative predictive value. In practice, classifier scores are compared against established threshold values, so during model evaluation, these metrics are assessed across multiple threshold values with the resulting quantifications of performance corresponding to these use cases. Threshold dependent metrics were evaluated across the domain of threshold values (0.1 to 0.9), for thresholds corresponding to commonly targeted sensitivity values (0.5, 0.9, 0.95), for thresholds corresponding to tolerant precision values (0.25, 0.33), as well as for thresholds which provide the maximum F1, F2, and Youden metrics.

At step 511, the method takes one or more actions in response to the calculated indication. For example, step 511 provides potential Organ Category Risk Scores derived from machine learning classifiers trained to assess how gene-centric features indicating tissue-specific activity, evolutionary conservation, localization, and disease-association affect Adverse Events (AEs) reported in the academic literature and clinical trials. In some examples, Risk scores and contributing features are provided and intended for initial hypothesis generation to provide direction for further investigation. While based on robust evidence across a range of human genetic data types, ground truth can be difficult to establish across any data type, especially for novel genetic targets where data is often lacking. However, validation on the ML models has been performed, and the Gene-level Risk Score and Organ Category Risk Scores as highlighted by the disclosed method can provide a useful starting point for investigating potential risks. In addition, scores may suggest a link in one Organ Category, but it is advised to also investigate the initial disease/diagnosis classification results in related Organ Categories. For example, the ML model in step 509 may estimate Psychiatric System as an Organ Category Risk but given the overlap between Psychiatric and Nervous System traits, Nervous System Risk effects would also be a fruitful starting point. Top features and feature groups supporting Organ Category Score classifications provide more specific starting points and can be reported in step 511 to provide useful starting points for further investigation.

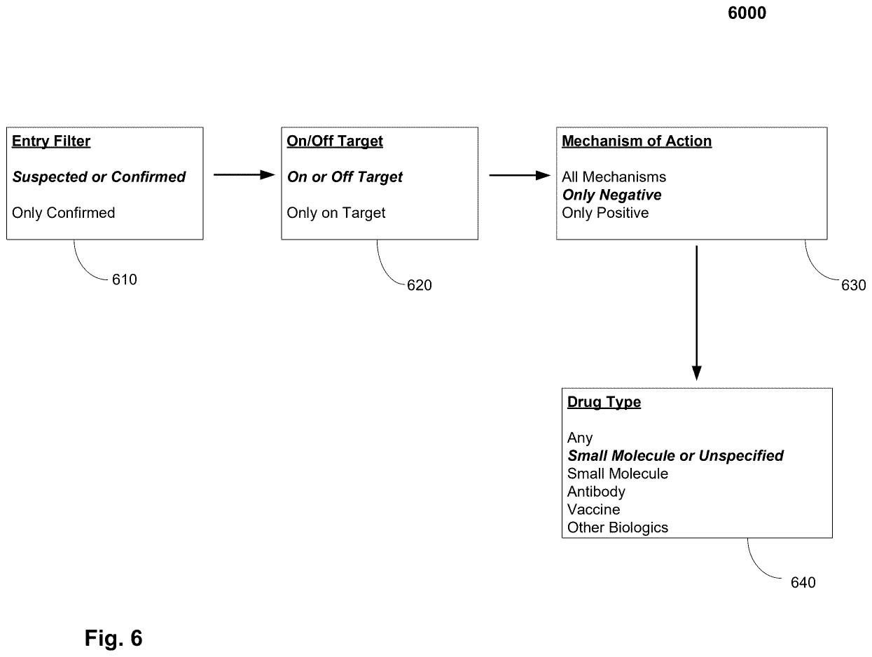

FIG. 6 shows an example flow diagram 6000 of filtering steps used to generate training labels for development of a machine learning model for a target risk assessment profile in accordance with an embodiment of the present disclosure. In some embodiments of the disclosed invention, the current combination of filters for the filtering steps 610-640 are designated in bold and italicized text in FIG. 6. Each filter shown in bold and italicized text is selected to, in combination, produce a classifier that estimates association of a gene target with likely adverse events in an organ category based on inhibitory modulation of its activity.

In some embodiments, the entry quality filter 610 removes entries which are less reliable based on the “level-of-evidence” label provided by a database of gene-centric known adverse events (AE) and human omics features. In one example implementation, existing models have included entries which are either “Confirmed” or “Suspected,” with “Suspected” being a label associated with less confident evidence. Alternative application of this filter could produce training labels which are more reliable, though at the cost of fewer entries and associations being available due to this higher evidentiary threshold. Adjusting the entry quality filter 610 does not produce classifiers with different scientific applications, just filtering based on the confidence of available data.

In some embodiments, the On/Off target filter 620 optionally restricts classification to drug-specific target indication associations (e.g., “On target”). In one example implementation, existing models have included entries with are labeled as either “On” or “Off” targets since in this example implementation, researchers are interested in all available evidence of drug modulation associated with reported adverse events, independent of a drug's intended application. Alternative application of this filter is effectively similar to the “level of evidence” filter for these applications since this label does not clarify any chemical or physiological association, just a description relative to existing knowledge of a drug. Adjusting this filter can produce classifiers with different design applications, though these require further altering the modeling approach and without those considerations, would produce classifiers that could be used for similar applications.

In some embodiments, the activity filter 630 is implemented by categorizing available mechanism of “action” entry annotations into broad categories of “activating” or “inhibiting”. In one example implementation, the training labels produced have included only entries which are categorized as “inhibiting” to produce classifiers which associates occurrence of adverse events which inhibit modulation of the gene target. Typically, gene activities have asymmetric tolerance of activity modulation, however significant inhibition or activation of activity can result in disrupted biological processes. Several gene targets are associated with different disease manifestations for activating versus inhibiting gene activity. Similarly, some drug interactions demonstrate different health outcomes depending on these modes of modulation. Classifiers used in one example embodiment can focus on inhibition because this can be easier to understand conceptually, and to design follow-up considerations for.

The systems and methods described herein allow for differentiation of whether the classifiers have learned “inhibition” versus “disruptive modulation” (which could include both inhibiting and activating effects). Adjusting this filter 630 produces labels which can train fundamentally different classifiers, pursuing adverse events association for genes based on inhibiting modulation, activating modulation, or any modulation. All of these applications can be useful, although in one current example implementation, the inhibiting labels/filter value is used.

In some embodiments, the drug type filter 640 separates database entries based on the chemical category of the drug in each entry. In one example implementation, training labels are developed for use in training the machine learning models, and the training database separates the drug types into “small molecule,” “vaccine,” or “biologic.” In one example implementation, for a particular drug, the database may lack this annotation (it is empty), e.g., categorization of “biologic” may not differentiate between categories of therapeutics. In such a scenario, a label of “antibody” can in some instances be reliably inferred from other columns containing drug information. One example implementation of the classifier utilizes entries which are “small molecules” or are un-annotated, since these are almost exclusively small molecules. Alternative applications of this filter produce fundamentally different classifiers since they are estimating association of adverse events from modulation by different drug types. There are clear future applications for such classifiers trained for “antibody” and “vaccine,” including combinations with the activity filter (consideration of inhibiting, activating, or both effects). Note that in this example implementation of the classifier, although implantation of filters does not require strict drug type values of “small molecule,” this difference does not alter the question addressed by classifiers trained on those differently curated different labels.

In some embodiments, using a similar downstream modeling approach, the entry quality and On/Off target filters adjust the specific quality and number of samples obtained, with their current values selected to produce the largest number of samples without consideration of low-quality entries. Both the activity filter and drug type filter each produce unique training labels that address fundamentally different scientific questions and applications. The activity filter as implemented can produce three different categories of labels. The drug type filter as implemented can produce four different categories of labels, three of which have clear applications (small molecule, antibody, vaccine). In one example implementation, the machine learning models are trained using labels corresponding to one of these nine possible applications. Other example embodiments of the disclosed invention are contemplated, where labels for these other eight possible applications can be generated and explored for future applications and comparison to existing classifiers. Explicitly, these combinations would be small molecule inhibition-which is the current machine learning model application. Other combinations include: small molecule activation; small molecule inhibition or activation; antibody inhibition; antibody activation; antibody inhibition or activation; vaccine inhibition; vaccine activation; vaccine inhibition or activation. In one current assessment, future applications of the other “biologic” category would require further annotations of these possible drug modalities.

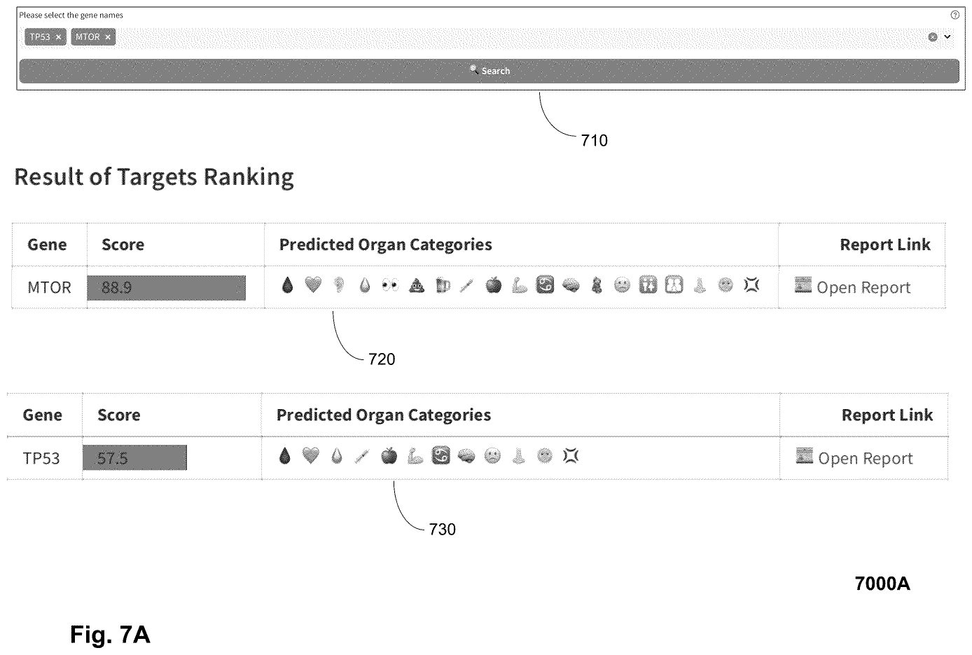

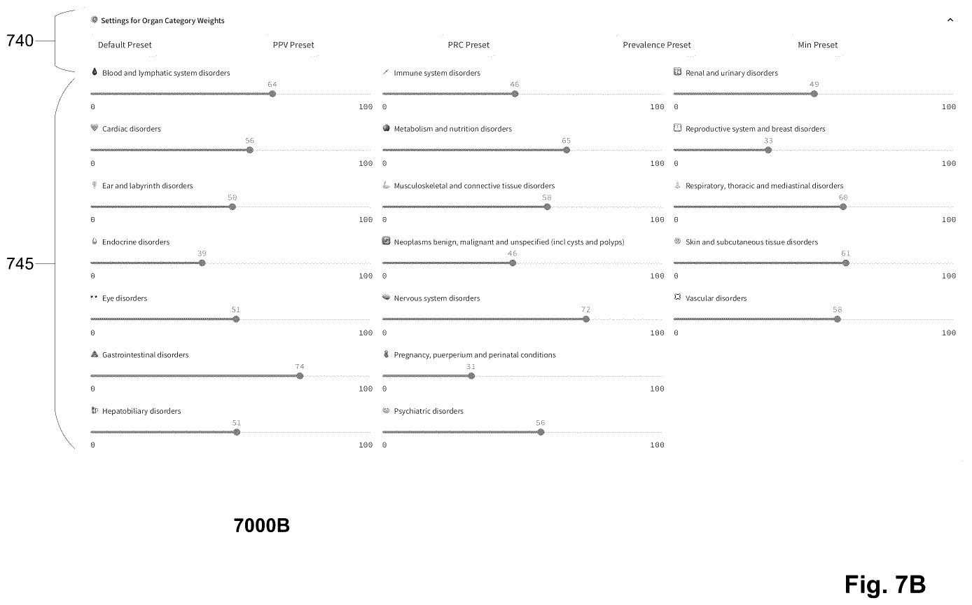

FIG. 7A shows an example of a user interface 7000A of an application for finding risk scores for one or more gene targets in accordance with an embodiment of the present disclosure. In some embodiments, user interface 7000A is used as part of a web application to find the risk score of a single gene-target or to compare the risk of a list of targets to determine which genes in a set of genes to prioritize for future drug research and/or development. Users can search for the risk scores of the target(s) by selecting the gene names in a drop-down menu. In some embodiments, the risk scores can be based on one or more machine learning models and/or algorithms as described above herein with respect to FIGS. 1-6. On user interface search bar 710, a user enters a single gene or list of genes in the search bar and presses search. Here in 710 the gene targets TP53 and MTOR are selected. The results are then displayed in results tables 720 and 730, which show the total risk scores for the gene target and the top organ categories identified by the machine learning models (e.g., “Estimated Risk Organ Categories”, meaning those organ categories with a greater than 0.5 risk score). In some embodiments, a user can hover over an emoji representing an Organ Category, which is listed under the “Estimated Risk Organ Categories” for the gene target MTOR in table 720 or the gene target TP53 in table 730, to view the associated organ category. Associated organ categories can also be found in a “Settings for Organ Category Weights” menu (e.g., the gear icon in element 740 in FIG. 7A. In some embodiments, more detailed individual gene score reports (not shown) can be generated and accessed via the user interface, e.g., by clicking on an “Open Report” link on the far right of tables 720 and 730.

FIG. 7B shows an example of a user interface 7000B for customizing risk scores for one or more gene targets in accordance with an embodiment of the present disclosure. In one embodiment, initial target scores are based on equal weighting of each organ category. Users can produce more bespoke risk scores by altering individual organ category weights in the “Settings for Organ Category Weights” menu which, in one embodiment, can be visible below the initially generated target ranking tables 720 and 730 of FIG. 7. On user interface 745 of FIG. 7A, weights can be adjusted manually by sliding the small circle underneath each organ category to the left (to decrease the weight) or to the right (to increase the weight).

In some embodiments, users can also adjust weights by using preset weighting schemes such as Positive Predictive Value (PPV), Positive Recall Curve (PRC), or Organ Category Risk Prevalence (Prevalence). These preset schemes can be selected by clicking on the appropriate button in user interface 740. The Min Preset button on interface 740 sets all scores to zero so that the user can then manually adjust individual Organ Category scores to focus on the most relevant organ categories related to expected local target(s) risks. Organ category risk prevalence (Prevalence) is the number of positive risk labels relative to the total number of positive and negative labels. PRC is the area under the precision-recall curve and single measure per organ category, characterizing the specificity of output scores. Minimum preset sets all weights to zero (equivalent to simple mean) so weights can be adjusted as needed. PPV is the number of true positives divided by the sum of the number of true positives and false positives and measures the precision of the machine learning model that is built and trained for the particular risk score measurements.

The risk score is calculated in some embodiments by the weighted sum of the scores of the organ categories. One example method to calculate the risk score is as follows:

$$Score = {\sum }_{i = 1}^{19} \frac{wi}{{\sum }_{j = 1}^{19} wj} \times si$$

where
   i and j are the index of the organ category,
   wi is the weight of the i-th organ category, which is adjustable by the user,

$${\sum }_{j = 1}^{19} wj$$

is the sum of weights for the 19 organ categories as specified by MedDRA, and
si is the score of the i-th organ category, derived from the ML model.

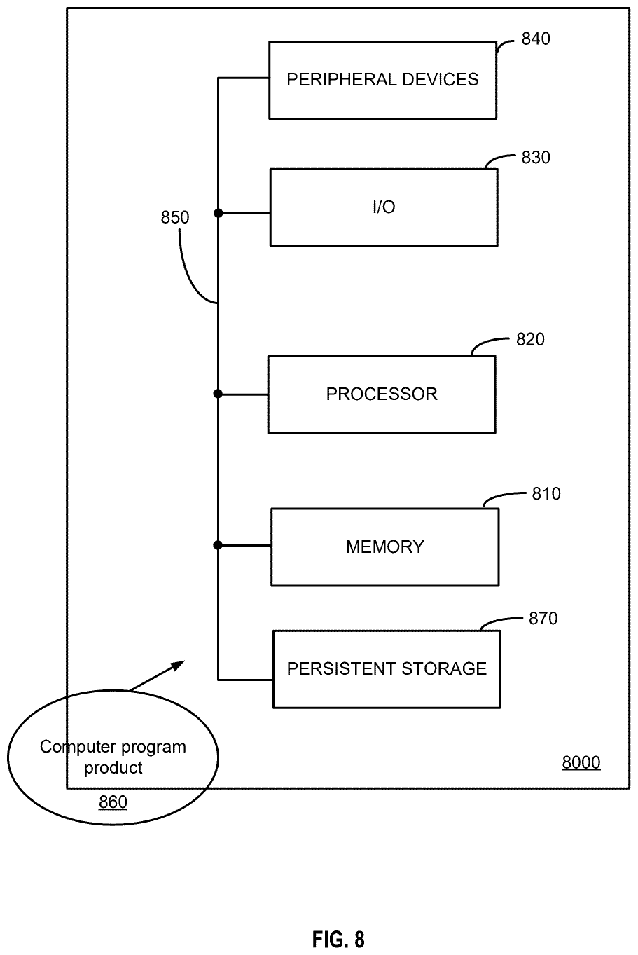

FIG. 8 shows an example of a computer system 8000, one or more of which may be used to implement one or more of the apparatuses, systems, and methods illustrated herein. Computer system 8000 executes instruction code contained in a computer program product 860. Computer program product 860 comprises executable code in an electronically readable medium that may instruct one or more computers such as computer system 8000 to perform processing that accomplishes the exemplary method steps performed.

The electronically readable medium may be any transitory or non-transitory medium that stores information electronically and may be accessed locally or remotely, for example via a network connection. The medium may include a plurality of geographically dispersed media, each configured to store various parts of the executable code at separate locations and/or at various times. The executable instruction code in an electronically readable medium directs the illustrated computer system 8000 to carry out various exemplary tasks described herein. The executable code for directing the carrying out of tasks described herein would be typically realized in software. However, it will be appreciated by those skilled in the art that computers or other electronic devices might utilize code realized in hardware to perform many or all the identified tasks. Those skilled in the art will understand that many variations on executable code may be found that implement exemplary methods within the spirit and the scope of the disclosure.

The code or a copy of the code contained in computer program product 560 may reside in one or more storage persistent media (not separately shown) communicatively coupled to system 8000 for loading and storage in persistent storage device 870 and/or memory 810 for execution by processor 820. Computer system 8000 also includes I/O subsystem 830 and peripheral devices 840. I/O subsystem 830, peripheral devices 840, processor 820, memory 810, and persistent storage device 870 are coupled via bus 850. Like persistent storage device 870 and any other persistent storage that might contain computer program product 860, memory 810 is a non-transitory medium (even if implemented as a typical volatile computer memory device). Moreover, those skilled in the art will appreciate that in addition to storing computer program product 860 for carrying out processing described herein, memory 810 and/or persistent storage device 870 may be configured to store the various data elements referenced and illustrated herein.

Those skilled in the art will appreciate computer system 8000 illustrates just one example of a system in which a computer program product in accordance with the disclosure may be implemented. To cite but one example, execution of instructions contained in a computer program product may be distributed over multiple computers, such as, for example, over the computers of a distributed computing network.

Instructions for implementing an artificial neural network or other deep learning network may reside in computer program product 860. When processor 820 is executing the instructions of computer program product 860, the instructions, or a portion thereof, are typically loaded into working memory 810 from which the instructions are readily accessed by processor 820.

Processor 820 may comprise multiple processors which may comprise respective additional working memories (additional processors and memories not individually illustrated) including one or more graphics processing units (GPUs) comprising at least thousands of arithmetic logic units supporting parallel computations on a large scale. GPUs are often utilized in deep learning applications because they can perform the relevant processing tasks more efficiently than can typical general-purpose processors (CPUs). Processor 820 may additionally or alternatively comprise one or more specialized processing units comprising systolic arrays and/or other hardware arrangements that support efficient parallel processing. Such specialized hardware may work in conjunction with a CPU and/or GPU to carry out the various processing described herein. Such specialized hardware may comprise application specific integrated circuits and the like (which may refer to a portion of an integrated circuit that is application-specific), field programmable gate arrays and the like, or combinations thereof. However, a processor such as processor 820 may be implemented as one or more general purpose processors (preferably having multiple cores) without necessarily departing from the spirit and scope of the present disclosure.

It should be apparent to those skilled in the art that many more modifications besides those already described are possible without departing from the inventive concepts herein. The inventive subject matter, therefore, is not to be restricted except as set forth in the appended claims. Moreover, in interpreting both the specification and the claims, all terms should be interpreted in the broadest possible manner consistent with the context. In particular, the terms “comprise” and “comprising” should be interpreted as referring to elements, compounds, or steps in a non-exclusive manner, indicating that the referenced elements, components, or steps are present, or utilized, or combined with other elements, components, or steps that are not expressly referenced. Where the specification or claims refer to at least one of something selected from the group consisting of A, B, C, . . . and N, the text should be interpreted as requiring only one element from the group, not A plus N, or B plus N, etc.

In some embodiments, numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. The numerical values presented in some embodiments of the invention can contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of any Markush groups used in the appended claims.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise one or more processors, such as a general-purpose processor, or an application specific integrated circuit (ASIC) configured to execute software instructions stored on a tangible, non-transitory computer readable storage medium (e.g., hard drive, solid state drive, RAM, flash, ROM, PLA, PLD, FPGA, etc.). The software instructions preferably configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps. In especially preferred embodiments, the various servers, systems, databases, or interfaces exchange data using standardized protocols or algorithms, possibly based on HTTP, HTTPS, AES, public-private key exchanges, web service APIs, or other electronic information exchanging methods. Data exchanges preferably are conducted over a packet-switched network, the Internet, LAN, WAN, VPN, or other type of packet switched network; a circuit switched network; cell switched network, or other type of network.

The above discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used in the description herein and throughout the claims that follow, when a system, engine, module, device, server, processor or other computing element is described as configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory thereby forming a structure having a specific purpose.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

While the present disclosure has been particularly described with respect to the illustrated embodiments, it will be appreciated that various alterations, modifications, and adaptations may be made based on the disclosure and are intended to be within the scope of the disclosure. While the disclosure has been described in connection with what are presently considered to be the most practical and preferred embodiments, it is to be understood that the present disclosure is not limited to the disclosed embodiments but, on the contrary, is intended to cover various modifications and equivalent arrangements included within the scope of the underlying principles as described by the various embodiments referenced above and below.

### ADDITIONAL EXAMPLE EMBODIMENTS

Example 1. A method of estimating a risk associated with patient safety, configured to execute on one or more computers, the method comprising:
obtaining a training data set comprising (a) drug-associated patient safety data, and (b) human omics data;
identifying one or more probabilistic features from the training data set, wherein the one or more probabilistic features comprise human omics features associated with a drug therapeutic;
deriving a ground truth label comprising a positive or a negative patient safety outcome corresponding to the one or more probabilistic features of the human omics data, wherein the ground truth label is associated with the drug therapeutic;
training one or more machine learning models using the training data set in accordance with the ground truth label; and
applying the trained one or more machine learning models to a patient dataset of human omics data to generate a calculated indication of a positive or a negative patient safety outcome.

Example 2. The method of example 1, wherein the drug-associated patient safety data comprises drug-associated known adverse event data.

Example 3. The method of example 1, wherein the calculated indication is a score indicating a risk of a positive or a negative patient safety outcome.

Example 4. The method of example 1, wherein the training data set of human omics data comprises human genomic data.

Example 5. The method of example 4, wherein the calculated indication of the positive or negative patient safety outcome comprises a risk level of a patient safety outcome associated with any of a plurality of organ classes related to the human genomic data.

Example 6. The method of example 4, wherein the calculated indication of the positive or negative patient safety outcome comprises a risk level of a patient safety outcome associated with a pre-specified organ class related to the human genomic data.

Example 7. The method of example 1, wherein the human omics data comprises one or more of gene constraint metrics, variant and gene-level phenome-wide association statistics, variant counts, essentiality scores, tolerance to loss of function metrics, gene expression data, and protein interaction network properties.

Example 8. The method of example 1, wherein the one or more human omics features comprise one or more gene-level features, network-level features, tissue-level features, disease-level features, or perturbation-level features.

Example 9. The method of example 1, wherein the one or more actions comprise a priority ranking of two or more drug targets based on patient safety.

Example 10. The method of example 1, wherein the one or more actions comprise a de-risking of at least one drug target in an early pre-clinical stage based on the calculated indication.

Example 11. The method of example 1, wherein the machine learning model of the one or more machine learning models is trained using training data set data comprising known adverse event data for a pre-specified organ class and generates a calculated indication of a positive or a negative patient safety outcome for the pre-specified organ class related to use of the drug therapeutic.

Example 12. The method of example 11, wherein the calculated indication of a positive or a negative patient safety outcome comprises a combination or transformation of a plurality of calculated indications of a positive or a negative patient safety outcome derived from a plurality of machine learning models, wherein each machine learning model is trained using training data set data comprising known adverse event data for a different pre-specified organ class, and the combination or transformation comprises a weighted average or other linear combination.

Example 13. The method of example 12, further comprising receiving a selection from a user of one or more target genes for which to generate the calculated indication of a positive or a negative patient safety outcome.

Example 14. The method of example 12, further comprising receiving a selection from a user adjusting one or more weights of the plurality of calculated indications of a positive or negative patient safety outcomes for the different pre-specified organ classes.

Example 15. The method of example 12, wherein the selection from the user adjusting the one or more weights comprises using one or more preset weightings of the plurality of calculated indications of a positive or negative patient safety outcomes for the different pre-specified organ classes.

Example 16. The method of example 15, wherein the one or more preset weightings comprise one or more of an organ risk prevalence weighting, a positive recall score weighting, a minimum preset weighting, a positive predictive value (PPV) weighting, and a preset weighting derived using an optimized gene-level categorization or ranking from an external dataset.

Example 17. A non-transitory computer readable medium configured for storing one or more machine readable instructions that upon execution by one or more processors of one or more computers perform the following operations:
training at least one machine learning model using one or more databases comprising one or more target genes, one or more drug therapeutics associated with the one or more target genes, adverse event data associated with the one or more drug therapeutics and the one or more target genes, and a plurality of human omics features associated with the one or more target genes derived from human omics data; and
generating one or more calculated indications of a positive or a negative patient safety outcome for one or more gene targets by applying the trained one or more machine learning models to a patient dataset of human omics data.

Example 18. The non-transitory computer readable medium of example 17, wherein the machine learning model of the one or more machine learning models is trained using training data set data comprising known adverse event data for a pre-specified organ class and generates a calculated indication of a positive or a negative patient safety outcome for the pre-specified organ class related to use of the drug therapeutic.

Example 19. The non-transitory computer readable medium of example 17, wherein the calculated indication of a positive or a negative patient safety outcome comprises a combination or transformation of a plurality of calculated indications of a positive or a negative patient safety outcome derived from a plurality of machine learning models, wherein each machine learning model is trained using training data set data comprising known adverse event data for a different pre-specified organ class.

Example 20. The non-transitory computer readable medium of example 19, wherein the combination or transformation comprises a weighted average or other linear combination.

Example 21. The non-transitory computer readable medium of example 17, further comprising priority ranking of two or more gene targets based on patient safety.

Example 22. The non-transitory computer readable medium of example 17, further comprising de-risking of at least one gene target in an early pre-clinical stage based on the calculated indication.

Example 23. The non-transitory computer readable medium of example 22, wherein the selection from the user adjusting the one or more weights comprises using one or more preset weightings of the plurality of calculated indications of a positive or negative patient safety outcomes for the different pre-specified organ classes wherein the one or more preset weightings comprise one or more of an organ risk prevalence weighting, a positive recall score weighting, a minimum preset weighting, a positive predictive value (PPV) weighting, or a preset weighting derived using an optimized gene-level categorization or ranking from an external dataset.

### Example Features and Organ Categories

### Example Features:

‘Common Essential Gene’, ‘Apical Target’, ‘Secreted Protein’, ‘ECM Protein’, ‘Surface Protein’, ‘Surface protein: number of conditions’,
‘IH_none’, ‘IH_Uncertain’, ‘IH_Supported’, ‘IH_Approved’, ‘IH_Enhanced’,
‘ITier_score’, ‘ITier_none’, ‘ITier_1A’, ‘ITier_1B’, ‘ITier_2A’, ‘ITier_2B’, ‘ITier_2C’, ‘ITier_3’,
‘Internalization Combined Weight’, ‘Multi-Drug Resistance Factors’, ‘Drug metabolism-cytochrome P450’, ‘Drug metabolism—other enzymes’, ‘N critical over-expressors’, ‘N less critical over-expressors’, ‘N brain over-expressors’, ‘Median critical normal expression’, ‘Pan-cancer median’, ‘Antigen Selectivity’,
‘TEPG_Cardiovascular_median’, ‘TEPG_Connective tissue_median’, ‘TEPG_Digestive_median’, ‘TEPG_Endocrine_median’, ‘TEPG_Immune_median’, ‘TEPG_Muscle_median’, ‘TEPG_Nervous Central_median’, ‘TEPG_Nervous Peripheral median’, ‘TEPG_Reproductive_median’, ‘TEPG_Respiratory_median’, ‘TEPG_Skin_median’, ‘TEPG_Urinary_median’,
‘TEPGt_Cardiovascular_Heart. Ventricle_median’,
‘TEPGt_Cardiovascular_Heart.Atrial_Appendage_median’,
‘TEPGt_Cardiovascular_Artery.Coronary_median’,
‘TEPGt_Cardiovascular_Artery.Aorta_median’,
‘TEPGt_Cardiovascular_Artery. Tibial_median’,
‘TEPGt_Connective_tissue_Adipose.Subcutaneous_median’,
‘TEPGt_Connective_tissue_Adipose. Visceral_median’,
‘TEPGt_Digestive_Colon.Distal_median’,
‘TEPGt_Digestive_Esophagus.Mucosa_median’,
‘TEPGt_Digestive_Esophagus_median’,
‘TEPGt_Digestive_Esophagus.Muscularis_median’,
‘TEPGt_Digestive_Minor_Salivary_Gland_median’,
‘TEPGt_Digestive_Colon.Proximal_median’, ‘TEPGt_Digestive_Ileum_median’,
‘TEPGt_Digestive_Jejunum_median’, ‘TEPGt_Digestive_Stomach_median’,
‘TEPGt_Digestive_Pancreas_median’, ‘TEPGt_Digestive_Liver_median’,
‘TEPGt_Endocrine_Pineal_Gland_median’,
‘TEPGt_Endocrine_Pituitary_Gland_median’,
‘TEPGt_Endocrine_Pancreatic_Islet_median’,
‘TEPGt_Endocrine_Thyroid_Gland_median’,
‘TEPGt_Endocrine_Adrenal_Gland_median’, ‘TEPGt_Immune_Thymus_median’,
‘TEPGt_Immune_CD4_Th2_median’, ‘TEPGt_Immune_Lymph_Node_median’,
‘TEPGt_Immune_CD8_EM_median’, ‘TEPGt_Immune_CD4_Th17_median’,
‘TEPGt_Immune_CD8_CM_median’, ‘TEPGt_Immune_CD4_Th1_median’,
‘TEPGt_Immune_CD4_EM_median’, ‘TEPGt_Immune_Spleen_median’,
‘TEPGt_Immune_CD4_Treg_median’, ‘TEPGt_Immune_CD4_CM_median’,
‘TEPGt_Immune_CD8_Naive_median’, ‘TEPGt_Immune_B_CD5_median’,
‘TEPGt_Immune_CD4_Naive_median’, ‘TEPGt_Immune_B_Naive_median’,
‘TEPGt_Immune_B_Memory_median’, ‘TEPGt_Immune_Bone_Marrow_median’,
‘TEPGt_Immune_Whole_Blood_median’,
‘TEPGt_Muscle_Muscle.Skeletal_median’,
‘TEPGt_Muscle_Muscle.Smooth_median’,
‘TEPGt_Nervous_Central_NS08_Occipital_Lobe_median’,
‘TEPGt_Nervous_Central_NS04_Insula_median’,
‘TEPGt_Nervous_Central_NS04_Temporal_Lobe_median’,
‘TEPGt_Nervous_Central_NS03_Parietal_Lobe_median’,
‘TEPGt_Nervous_Central_NS10_Cerebellum_median’,
‘TEPGt_Nervous_Central_NS01_Olfactory_Bulb_median’,
‘TEPGt_Nervous_Central_NS03_Frontal_Cortex_median’,
‘TEPGt_Nervous_Central_NS03_Cingulate_Cortex_median’,
‘TEPGt_Nervous_Central_NS05_Hypothalamus_median’,
‘TEPGt_Nervous_Central_NS06_Thalamus_median’,
‘TEPGt_Nervous_Central_NS07_Pons_median’,
‘TEPGt_Nervous_Central_NS05_Amygdala_median’,
‘TEPGt_Nervous_Central_NS05_Nucleus_Accumbens_median’,
‘TEPGt_Nervous_Central_NS07_Hippocampus_median’,
‘TEPGt_Nervous_Central_NS06_Substantia_Nigra_median’,
‘TEPGt_Nervous_Central_NS03_Caudate_Nucleus_median’,
‘TEPGt_Nervous_Central_NS05_Putamen_median’,
‘TEPGt_Nervous_Central_NS09_Medulla_Oblongata_median’,
‘TEPGt_Nervous_Central_NS06_Habenula_median’,
‘TEPGt_Nervous_Central_NS12_Spinal_Cord_median’,
‘TEPGt_Nervous_Central_NS02_Cerebral_Meninges_median’,
‘TEPGt_Nervous_Peripheral_Nerve.Tibial_median’,
‘TEPGt_Reproductive_Testis_median’,
‘TEPGt_Reproductive_Mammary_Gland_median’,
‘TEPGt_Reproductive_Prostate_median’, ‘TEPGt_Reproductive_Placenta_median’,
‘TEPGt_Reproductive_Vagina_median’, ‘TEPGt_Reproductive_Cervix_median’,
‘TEPGt_Reproductive_Uterus_median’, ‘TEPGt_Reproductive_Ovary_median’,
‘TEPGt_Respiratory_Lung_median’, ‘TEPGt_Skin_Skin_median’,
‘TEPGt_Urinary_Bladder_median’, ‘TEPGt_Urinary_Kidney_median’,
‘TEPcF_blood_vessel endothelial cell_quorum’, ‘TEPcF_epithelial_cell_quorum’,
‘TEPcF_fibroblast_quorum’, ‘TEPcF_mesenchymal_precursor_cell_quorum’,
‘TEPcF_mesenchymal_stem_cell_quorum’, ‘TEPcF_preadipocyte_quorum’,
‘TEPcF_smooth_muscle_cell_quorum’,
‘TEPcFoc_blood_quorum’, ‘TEPcFoc_blood_vessel_quorum’,
‘TEPcFoc_breast_quorum’, ‘TEPcFoc_central_nervous_system_quorum’,
‘TEPcFoc_connective_tissue_quorum’, ‘TEPcFoc_embryo_quorum’,
‘TEPcFoc_female_reproductive_system_quorum’,
‘TEPcFoc_gastrointestinal_system_quorum’, ‘TEPcFoc_gum_quorum’,
‘TEPcFoc_heart_quorum’,
‘TEPcFoc_hematopoietic_and_lymphoid_system_quorum’, ‘TEPcFoc_liver_quorum’,
‘TEPcFoc_male_reproductive_system_quorum’, ‘TEPcFoc_muscle_quorum’,
‘TEPcFoc_respiratory system_quorum’, ‘TEPcFoc_sensory_system_quorum’,
‘TEPcFoc_skin_quorum’, ‘TEPcFoc_urinary_system_quorum’,
‘TEPcFt_blood_vessel_aorta_quorum’,
‘TEPcFt_central_nervous_system_brain_quorum’,
‘TEPcFt_central_nervous_system_spinal cord_quorum’,
‘TEPcFt_connective_tissue_adipose_tissue_quorum’,
‘TEPcFt_embryo_amnion_quorum’,
‘TEPcFt_hematopoietic_and_lymphoid_system_bone_marrow_quorum’,
‘TEPcFt_male_reproductive_system_prostate_quorum’, ‘TEPcFt_muscle_skeletal muscle_quorum’, ‘TEPcFt_respiratory_system_lung_quorum’,
‘TEPcFt_sensory_system_eye_quorum’, ‘TEPcFt_urinary_system_kidney_quorum’,
‘CMA_pLI’, ‘CMA_oe_lof’, ‘CMA_LOEUF’, ‘CMA_LOEUF_percentile’,
‘CMA_LoFTool_percentile’, ‘CMA_VIRLoF_percentile’, ‘CMA_GeVIR_percentile’,
‘CMA_RVIS_ExAC’, ‘CMA_RVIS_percentile_ExAC’, ‘CMS_pLI’, ‘CMS_oe_lof’,
‘CMS_LOEUF’, ‘CMS_LOEUF_percentile’, ‘CMS_LoFTool_percentile’,
‘CMS_VIRLoF_percentile’, ‘CMS_GeVIR_percentile’, ‘CMS_RVIS_ExAC’,
‘CMS_RVIS_percentile_ExAC’,
‘Picklesv3_Avana_Z’, ‘Picklesv3_Avana_BF’, ‘Picklesv3_Avana_Chronos’, ‘Picklesv3_Score_Z’, ‘Picklesv3_Score_BF’, ‘Picklesv3_TKOv3_Z’, ‘Picklesv3_TKOv3_BF’,
‘INH_AD’, ‘INH_AR’, ‘INH_ADAR’, ‘INH_XLD’, ‘INH_XLR’, ‘INH_XLother’, ‘INH_MT’, ‘INH_Smu’, ‘INH_multi’ ‘ns_snv_ukb_wes’,
‘ns_snv_ukb_imp’, ‘ns_snv_ukb_wgs140k’, ‘ns_common_ukb_wes’,
‘ns_common_ukb_imp’, ‘ns_common_ukb_wgs140k’, ‘ns_rare_ukb_wes’,
‘ns_rare_ukb_imp’, ‘ns_rare_ukb_wgs140k’, ‘ns_urare_ukb_wes’,
‘ns_urare_ukb_imp’, ‘ns_urare_ukb_wgs140k’, ‘ns_missense_ukb_wes’,
‘ns_missense_ukb_imp’, ‘ns_missense_ukb_wgs140k’, ‘ns_misscadd_ukb_wes’,
‘ns_misscadd_ukb_imp’, ‘ns_misscadd_ukb_wgs140k’, ‘ns_plof_ukb_wes’,
‘ns_plof_ukb_imp’, ‘ns_plof_ukb_wgs140k’, ‘ns_CVP_ukb_wes’, ‘ns_CVP_ukb_imp’,
‘ns_CVP_ukb_wgs140k’, ‘ns_CVLP_ukb_wes’, ‘ns_CVLP_ukb_imp’,
‘ns_CVLP_ukb_wgs140k’,
‘PHEWGSb_Blood_and_lymphatic_system_disorders_pos’,
‘PHEWGSb_Cardiac_disorders_pos’,
‘PHEWGSb_Congenital_familial_and_genetic_disorders_pos’,
‘PHEWGSb_Ear_and_labyrinth_disorders_pos’,
‘PHEWGSb_Endocrine_disorders_pos’, ‘PHEWGSb_Eye_disorders_pos’,
‘PHEWGSb_Gastrointestinal_disorders_pos’,
‘PHEWGSb_General_disorders_and_administration_site_conditions_pos’,
‘PHEWGSb_Hepatobiliary_disorders_pos’,
‘PHEWGSb_Immune_system_disorders_pos’,
‘PHEWGSb_Infections_and_infestations_pos’,
‘PHEWGSb_Injury_poisoning_and_procedural_complications_pos’,
‘PHEWGSb_Investigations_pos’,
‘PHEWGSb_Metabolism_and_nutrition_disorders_pos’,
‘PHEWGSb_Musculoskeletal_and_connective_tissue_disorders_pos’,
‘PHEWGSb_Neoplasms_benign_malignant_and_unspecified_pos’,
‘PHEWGSb_Nervous_system_disorders_pos’,
‘PHEWGSb_Pregnancy_puerperium_and_perinatal_conditions_pos’,
‘PHEWGSb_Psychiatric_disorders_pos’,
‘PHEWGSb_Renal_and_urinary_disorders_pos’,
‘PHEWGSb_Reproductive_system_and_breast_disorders_pos’,
‘PHEWGSb_Respiratory_thoracic_and_mediastinal_disorders_pos’,
‘PHEWGSb_Skin_and_subcutaneous_tissue_disorder_pos’,
‘PHEWGSb_Surgical_and_medical_procedures_pos’,
‘PHEWGSb_Vascular_disorders_pos’,
‘PAIqtb_blood_pos_beta’, ‘PAIqtb_blood_pos_log 10p’, ‘PAIqtb_cardiac_pos_beta’,
‘PAIqtb_cardiac_pos_log 10p’, ‘PAIqtb_eye_pos_beta’, ‘PAIqtb_eye_pos_log 10p’,
‘PAIqtb_renal_pos_beta’, ‘PAIqtb_renal_pos_log 10p’, ‘PAIqtb_resp_pos_beta’,
‘PAIqtb_resp_pos_log 10p’,
‘PAICIc_respiratory_respiratory’, ‘PAICIc_genitourinary_genitourinary’,
‘PAICIc_hematopoietic_hematopoietic’,
‘PAICIc_circulatory_system_circulatory_system’,
‘PAICIc_dermatologic_dermatologic’, ‘PAICIc_musculoskeletal_musculoskeletal’,
‘PAICIc_digestive_digestive’, ‘PAICIc_neurological_neurological’,
‘PAICIc_endocrine/metabolic_endocrine/metabolic’,
‘PAICImr_respiratory_respiratory_pos_beta’,
‘PAICImr_respiratory_respiratory_pos_pval’,
‘PAICImr_respiratory_respiratory_neg_beta’,
‘PAICImr_respiratory_respiratory_neg_pval’,
‘PAICImr_genitourinary_genitourinary_pos_beta’,
‘PAICImr_genitourinary_genitourinary_pos_pval’,
‘PAICImr_genitourinary_genitourinary_neg_beta’,
‘PAICImr_genitourinary_genitourinary_neg_pval’,
‘PAICImr_hematopoietic_hematopoietic_pos_beta’,
‘PAICImr_hematopoietic_hematopoietic_pos_pval’,
‘PAICImr_hematopoietic_hematopoietic_neg_beta’,
‘PAICImr_hematopoietic_hematopoietic_neg_pval’,
‘PAICImr_circulatory_system_circulatory_system_pos_beta’,
‘PAICImr_circulatory_system_circulatory_system_pos_pval’,
‘PAICImr_circulatory_system_circulatory_system_neg_beta’,
‘PAICImr_circulatory_system_circulatory_system_neg_pval’,
‘PAICImr_dermatologic_dermatologic_pos_beta’,
‘PAICImr_dermatologic_dermatologic_pos_pval’,
‘PAICImr_dermatologic_dermatologic_neg_beta’,
‘PAICImr_dermatologic_dermatologic_neg_pval’,
‘PAICImr_musculoskeletal_musculoskeletal_pos_beta’,
‘PAICImr_musculoskeletal_musculoskeletal_pos_pval’,
‘PAICImr_musculoskeletal_musculoskeletal_neg_beta’,
‘PAICImr_musculoskeletal_musculoskeletal_neg_pval’,
‘PAICImr_digestive_digestive_pos_beta’, ‘PAICImr_digestive_digestive_pos_pval’,
‘PAICImr_digestive_digestive_neg_beta’, ‘PAICImr_digestive_digestive_neg_pval’,
‘PAICImr_neurological_neurological_pos_beta’,
‘PAICImr_neurological_neurological_pos_pval’,
‘PAICImr_neurological_neurological_neg_beta’,
‘PAICImr_neurological_neurological_neg_pval’,
‘PAICImr_endocrine/metabolic_endocrine/metabolic_pos_beta’,
‘PAICImr_endocrine/metabolic_endocrine/metabolic_pos_pval’,
‘PAICImr_endocrine/metabolic_endocrine/metabolic_neg_beta’,
‘PAICImr_endocrine/metabolic_endocrine/metabolic_neg_pval’,

### Example Gene Network Features:

```
net_feat_cols =[
  ‘net_degree_centrality’, ‘net_betweenness_centrality’,
  ‘net_closeness_centrality’, ‘net_eigenvector_centrality’,
  ‘net_number_edges’, ‘net_clustering_coefficient’, ‘net_has_self_loop’,
  ‘net_phe_pos_circulatory_system’, ‘net_phe_pos_dermatologic’,
  ‘net_phe_pos_digestive’, ‘net_phe_pos_endocrine/metabolic’,
  ‘net_phe_pos_genitourinary’, ‘net_phe_pos_hematopoietic’,
  ‘net_phe_pos_mental_disorders’, ‘net_phe_pos_musculoskeletal’,
  ‘net_phe_pos_neoplasms’, ‘net_phe_pos_neurological’,
  ‘net_phe_pos_pregnancy_complications’, ‘net_phe_pos_respiratory’,
  ‘net_phe_pos_sense_organs’, ‘net_phe_neg_circulatory_system’,
  ‘net_phe_neg_dermatologic’, ‘net_phe_neg_digestive’,
  ‘net_phe_neg_endocrine/metabolic’, ‘net_phe_neg_genitourinary’,
  ‘net_phe_neg_hematopoietic’, ‘net_phe_neg_mental_disorders’,
  ‘net_phe_neg_musculoskeletal’, ‘net_phe_neg_neoplasms’,
  ‘net_phe_neg_neurological’, ‘net_phe_neg_pregnancy_complications’,
  ‘net_phe_neg_respiratory’, ‘net_phe_neg_sense_organs’,
  ‘net_plof_pos_circulatory_system’, ‘net_plof_pos_dermatologic’,
  ‘net_plof_pos_digestive’, ‘net_plof_pos_endocrine/metabolic’,
  ‘net_plof_pos_genitourinary’, ‘net_plof_pos_hematopoietic’,
  ‘net_plof_pos_mental_disorders’, ‘net_plof_pos_musculoskeletal’,
  ‘net_plof_pos_neoplasms’, ‘net_plof_pos_neurological’,
  ‘net_plof_pos_pregnancy_complications’, ‘net_plof_pos_respiratory’,
  ‘net_plof_pos_sense_organs’, ‘net_plof_neg_circulatory_system’,
  ‘net_plof_neg_dermatologic’, ‘net_plof_neg_digestive’,
  ‘net_plof_neg_endocrine/metabolic’, ‘net_plof_neg_genitourinary’,
  ‘net_plof_neg_hematopoietic’, ‘net_plof_neg_mental_disorders’,
  ‘net_plof_neg_musculoskeletal’, ‘net_plof_neg_neoplasms’,
  ‘net_plof_neg_neurological’, ‘net_plof_neg_pregnancy_complications’,
  ‘net_plof_neg_respiratory’, ‘net_plof_neg_sense_organs’
  ]
```

### Example Organ Categories:

```
off_x_targets=[
  ‘OC.Blood and lymphatic system disorders’,
  ‘OC.Cardiac disorders’,
  ‘OC.Ear and labyrinth disorders’, ‘OC.Endocrine disorders’,
  ‘OC.Eye disorders’, ‘OC.Gastrointestinal disorders’,
  ‘OC.Hepatobiliary disorders’, ‘OC.Immune system disorders’,
  ‘OC.Metabolism and nutrition disorders’,
  ‘OC.Musculoskeletal and connective tissue disorders’,
  ‘OC.Neoplasms benign, malignant and unspecified (incl cysts and polyps)’,
  ‘OC.Nervous system disorders’,
  ‘OC.Pregnancy, puerperium and perinatal conditions’,
  ‘OC.Psychiatric disorders’, ‘OC.Renal and urinary disorders’,
  ‘OC.Reproductive system and breast disorders’,
  ‘OC.Respiratory, thoracic and mediastinal disorders’,
  ‘OC.Skin and subcutaneous tissue disorders’, ‘OC.Vascular disorders’
]
```

What is claimed is:

1. A computer-executable deep learning network stored in a non-transitory computer readable medium and configured to execute on one or more processors of one or more computers to estimate a patient safety risk comprising:
a training data set comprising (a) drug-associated patient safety data, and (b) human omics data;
a feature space having one or more probabilistic features wherein the one or more probabilistic features comprise one or more human omics features associated with a drug therapeutic; and
a pre-trained machine learning analyzer comprising one or more machine learning models configured to receive one or more gene targets and generate one or more calculated indications, wherein the pre-training of the machine learning analyzer comprises one or more operations including deriving a ground truth label comprising a positive or a negative patient safety outcome corresponding to the one or more probabilistic features of the human omics data, wherein the ground truth label is associated with the drug-associated patient safety data of the drug therapeutic; and training one or more machine learning models using the training data set in accordance with the ground truth label.

2. The computer-executable deep learning network according to claim 1 wherein the machine learning analyzer comprises a gradient boosting machine.

3. The computer-executable deep learning network according to claim 1 wherein the machine learning analyzer comprises a logistic regression-based analyzer.

4. The computer-executable deep learning network according to claim 1 wherein the machine learning analyzer comprises a machine learning classifier and the one or more risk scores comprise one or more classifications expressed as a class probability.

5. The computer-executable deep learning network according to claim 1, wherein the calculated indication is a score indicating a risk of a positive or a negative patient safety outcome.

6. The computer-executable deep learning network according to claim 1, wherein the training data set of human omics data comprises human genomic data.

7. The computer-executable deep learning network according to claim 6, wherein the calculated indication of the positive or negative patient safety outcome comprises a risk level of a patient safety outcome associated with any of a plurality of organ classes related to the human genomic data.

8. The computer-executable deep learning network according to claim 6, wherein the calculated indication of the positive or negative patient safety outcome comprises a risk level of a patient safety outcome associated with a pre-specified organ class related to the human genomic data.

9. The computer-executable deep learning network according to claim 1, wherein the human omics data comprises one or more of gene constraint metrics, variant and gene-level phenome-wide association statistics, variant counts, essentiality scores, tolerance to loss of function metrics, gene expression data, and protein interaction network properties.

10. The computer-executable deep learning network according to claim 1, wherein the one or more human omics features comprise one or more gene-level features, network-level features, tissue-level features, disease-level features, or perturbation-level features.

11. The computer-executable deep learning network according to claim 1, further comprising an application interface configured to receive a user request and output a priority ranking of two or more gene targets based on patient safety.

12. The computer-executable deep learning network according to claim 1, further comprising an application interface configured to receive a user request and output a de-risking of at least one gene target in an early pre-clinical stage based on the calculated indication.

13. The computer-executable deep learning network according to claim 1, wherein the machine learning model of the one or more machine learning models is trained using training data set data comprising known adverse event data for a pre-specified organ class and generates a calculated indication of a positive or a negative patient safety outcome for the pre-specified organ class related to use of the drug therapeutic.

14. The computer-executable deep learning network according to claim 13, wherein the calculated indication of a positive or a negative patient safety outcome comprises a combination or transformation of a plurality of calculated indications of a positive or a negative patient safety outcome derived from a plurality of machine learning models, wherein each machine learning model is trained using training data set data comprising known adverse event data for a different pre-specified organ class.

15. A non-transitory computer readable medium configured for storing one or more machine readable instructions that upon execution by one or more processors of one or more computers perform the following operations:
training at least one machine learning model using one or more databases comprising one or more target genes, one or more drug therapeutics associated with the one or more target genes, adverse event data associated with the one or more drug therapeutics and the one or more target genes, and a plurality of human omics features associated with the one or more target genes derived from human omics data; and
generating one or more calculated indications of a positive or a negative patient safety outcome for one or more gene targets by applying the trained one or more machine learning models to a patient dataset of human omics data.

16. The non-transitory computer readable medium of claim 15, wherein the calculated indication of the positive or negative patient safety outcome comprises a risk level of a patient safety outcome associated with any of a plurality of organ classes related to the human genomic data.

17. The non-transitory computer readable medium of claim 15, wherein the calculated indication of the positive or negative patient safety outcome comprises a risk level of a patient safety outcome associated with a pre-specified organ class related to the human genomic data.

18. The non-transitory computer readable medium of claim 15, wherein the operations further comprise receiving a selection from a user of one or more target genes for which to generate the calculated indication of a positive or a negative patient safety outcome.

19. The non-transitory computer readable medium of claim 15, further comprising receiving a selection from a user adjusting one or more weights of the plurality of calculated indications of a positive or negative patient safety outcomes for the different pre-specified organ classes.

20. A computer-implemented method of estimating a risk associated with patient safety, the method comprising:
generating one or more calculated indications of a positive or a negative patient safety outcome for one or more gene targets by applying one or more pre-trained machine learning models to a patient dataset of human omics data,
wherein the one or more pre-trained machine learning models are trained using one or more databases comprising one or more target genes, one or more drug therapeutics associated with the one or more target genes, adverse event data associated with the one or more drug therapeutics and the one or more target genes, and a plurality of human omics features associated with the one or more target genes, the plurality of human omics features derived from human omics data.
